(19) United States
(12) Patent Application Publication
(10) Pub. No.: US 2013/0198981 A1
(43) Pub. Date: Aug. 8, 2013

(54) Gutter Inspection and Cleaning Apparatus

(71) Applicant: Nalian Ainsworth Crawford, Douglasville, GA (US)

(72) Inventor: Nalian Ainsworth Crawford, Douglasville, GA (US)

(21) Appl. No.: 13/367,372

(22) Filed: Feb. 7, 2012

(51) Int. Cl. E04D 13/076; G01N 33/00

(52) U.S. Cl. 15/93.1; 73/865.8

(57) ABSTRACT

This is a rain gutter inspection and cleaning apparatus that is affixed to a hollow telescopic electric pole (herein referred to as the handle) that removes leaves, pine needled and other debris from a rain gutter, thus allowing water to flow unobstructed to the downspout. The said tool can be operated while standing on the ground or while propped-up on a step assist. This tool is unique in that, it allows the user to also clean the gutter while standing on the roof. A power mirror attachment is optimally affixed above the scraper plate, allowing the user to view the debris collected in the gutter from the ground level. This gutter inspection and cleaning apparatus uses two types of mirrors. There is a power mirror that can be adjusted manually or remotely using a joystick. There also a non-powered manually operated mirror attachment, which is interchangeable with the power mirror attachment on the said gutter inspection and cleaning apparatus. The inspection of a gutter using this gutter inspection and cleaning apparatus, can be carried out before, during and after the cleaning operation.

### BACKGROUND OF THE INVENTION

This invention relates to a home improvement gutter inspection and cleaning apparatus that is used by a home owner in properly maintaining a rain gutter, or network of gutters, which serves the purpose of diverting water away from the foundation of a building or structure in a controlled manner. This apparatus will instill self-reliance in homeowners who are normally responsible for the maintenance of their homes or properties. This self reliance is likely to result in an increase in the preventative maintenance, as it relates to the gutter cleaning schedule. This inspection and cleaning apparatus is especially suited for low, to medium or high pitch roof on a single or multi-story building where the gutters are easily accessible.

### PRIOR ART

Rain gutter cleaning tools, device, equipment etc. have been around for generations. As our population grows and home ownership increases, there is an increasing need for an economically viable gutter inspection and cleaning apparatus. This is especially the case now that more and more individuals seek to protect their investments.

In doing so, one would think homeowners would become more and more reliant on gutter cleaning tools for protecting their investments. But unfortunately, this is not the case. The reality of the situation is that there is not enough if any, easy to use, light weight, easy to store, economically viable (low cost), and user friendly gutter inspection and cleaning apparatus that is available for purchase at the local hardware store. The present aforementioned design has culminated all the key features referenced above into one inspection and cleaning apparatus that is a delight to the homeowner.

The design of the aforementioned gutter inspection and cleaning apparatus is meant to solve the short comings that have plagued many of the designs that have been patented over the past several decades. In the previous works that have been presented, the use of mirrors have been employed, but are not well integrated into the design to maximize the usefulness of the apparatus as is the case of this present art. A key feature of the aforementioned gutter inspection and cleaning apparatus is that the mirror is power operated and covers several incremental viewing angles.

In prior arts, the mirror used, lack the viewing angles and the degrees of freedom generated by the ball joint mechanism that is presented herein. The operation of the power mirror attachment can be carried out by the user while the inspection and cleaning apparatus is moving or stationary in the gutter. In prior arts, the user would have to remove the apparatus from the overhead gutter to carry out any necessary adjustment of the mirror, in order to optimize the viewing angles of the debris. This process may have to be repeated several times which can be frustrating and most of all, time consuming. The degrees of freedom are achieved through translations and rotational movements.

This gutter inspection and cleaning apparatus in addition to the power mirror attachment, as a feature which allows it to be used with a non-powered fully manually operated mirror attachment mounted to a mirror housing retainer. This quality affordable and lower cost option is based on the same principles as the power mirror attachment version. No other prior art offers both types of mirror attachment options.

In prior arts that use clamps, gripper arms, scoops et al, precise care and maneuvering, involving clamping pressure are required before any debris can be removed. This too can be frustrating, as it may take several repeated attempts for the optimal clamping pressure to be achieved in order to retrieve smaller pieces of debris from the gutter. The fact that this design uses a spring loaded single scraper mechanism makes it easy to remove debris in a single forward pull of the telescoping electric pole—in a direction parallel to the surface of the gutter. After each stroke or pull of the handle, the scraper plate which is part of the scraper mechanism, will return to its rest position, as the inspection and cleaning apparatus is lifted inside the gutter in preparation to start the next cleaning cycle.

Another short coming with prior arts is that they do not allow the user to cleaning the gutter while standing on the roof, or through a second storey room window. This gutter inspection and cleaning apparatus as a dual cleaning mode, which presents the user with an additional user preference as mentioned above. In other words, this apparatus allows the user to clean the gutter while standing below or above the level of the gutter. None of the prior arts including, but not limited to U.S. Pat. Nos. 6,290,937, 6,925,676, 5,853,209, 5,855,402, 5,988,715, 6,017,070, 6,139,077, 7,802,338, 7,740,296, gives the user this ability. The versatility that is offered by this inspection and cleaning apparatus is second to none, thus making it a stand out when compared to any other apparatus in this category.

### SUMMARY OF THE INVENTION

The key to the uniqueness of this invention is its user friendliness, which is achieved through the use of light weight construction material, i.e. plastics. This makes the apparatus herein a perfect fit for the average home owner or professional gutter cleaner, whether male or female. The total weight of the apparatus without the hollow telescoping electric pole is less than 5 kg.

The spring loaded scraper mechanism is like no other in that, its design allows for the easy and almost effortless removal of debris from a rain gutter. This is achieved through the spring return system that allows the scraper plate to return to its rest position after completing a cleaning cycle. No manual actuation or reset of the scraper mechanism is required to start the next cleaning cycle.

The joystick and power mirror attachment mirror, makes up an integral part of this invention. This is a key feature of the invention as it adds value, and makes for an efficient inspection process. The user simple tugs on the joystick in either one of four directions to achieve the desired viewing angle. There is also an option for non-powered manual mirror attachment that is also available, as a direct fit to this apparatus. The reflective surface on the non-powered manual mirror attachment maneuvers and function in a similar manner to the powered mirror attachment about the ball joint.

Another unique feature of this gutter inspection and cleaning apparatus is its dual function, which gives it the ability to clean overhead gutters, or gutters directly at or below the feet. This adaptability is due to the two (2) headed neck design. To clean an overhead gutter, the user simply screws the hollow telescoping electric pole into head number 1 and proceeds with the inspection and cleaning operation. Subsequently, if the cleaning application requires cleaning a gutter at or below the feet level, the user simply screws the hollow telescopic electric pole unto head number 2, and proceeds with the cleaning operation. For cleaning a gutter at the ground level, the apparatus can either be used with or without the mirror attachments.

The teeth assembly is of a flexible spring construction design that allows for superb cleaning ability. When the gutter inspection and cleaning apparatus is pulled inside the gutter, the flexible spring like teeth assembly cleans, as it flexes to trap debris which can then be pulled over the side of the gutter.

### BRIEF DESCRIPTION OF THE DRAWINGS

To comprehend the design features of this gutter inspection and cleaning apparatus, several drawing views are presented;

FIG. 1 is a front view of the gutter inspection and cleaning apparatus.

FIG. 2 is a top or plan view of the gutter inspection and cleaning apparatus

FIG. 3 is a side view of the gutter inspection and cleaning apparatus

FIG. 4 is a top or plan view of the teeth assembly

FIG. 5 is a side view of the teeth assembly

FIG. 6 is a side view of the gutter inspection and cleaning apparatus, with the hollow telescoping electric pole attached to head number 2, and the scraper plate shown in the cleaning position

FIG. 7 is a top or plan view of the non-powered manual mirror attachment

FIG. 8 is a side view of the non-powered manual mirror attachment

FIG. 9 is a front view of the non-powered manual mirror attachment

FIG. 10 is a cross section of the hollow telescoping electric pole showing the 12V battery pack

FIG. 11 is a front view of the gutter inspection and cleaning apparatus, showing the reflective surface of the power mirror attachment tilted down to the left

FIG. 12 is a front view of the gutter inspection and cleaning apparatus, showing the reflective surface of the power mirror attachment tilted up to the left

FIG. 13 is a side view of the gutter inspection and cleaning apparatus, showing the reflective surface of the power mirror attachment tilted up to the left

FIG. 14 is a front view of the gutter inspection and cleaning apparatus, showing the power mirror attachment tilted about the ball joint to the right

FIG. 15 is a side view of the gutter inspection and cleaning apparatus, showing the power mirror attachment tilted down to the right, about the ball joint on the cantilever arm.

FIG. 16 is a top view of the gutter inspection and cleaning apparatus, showing the manual mirror attachment rotated to the right about the ball joint on the cantilever arm

FIG. 17 is a top view of the gutter inspection and cleaning apparatus, showing the power mirror attachment rotated to the right about the ball joint on the cantilever arm

FIG. 18 is a front view showing the manual mirror attachment tilted up to the left, about the ball joint on the cantilever arm

FIG. 19 is a side view showing the manual mirror attachment tilted up to the right about the ball joint on the cantilever arm

FIG. 20 shows the cantilever arm with the power mirror attachment tilted downwards

### DETAIL DESCRIPTION OF THE INVENTION

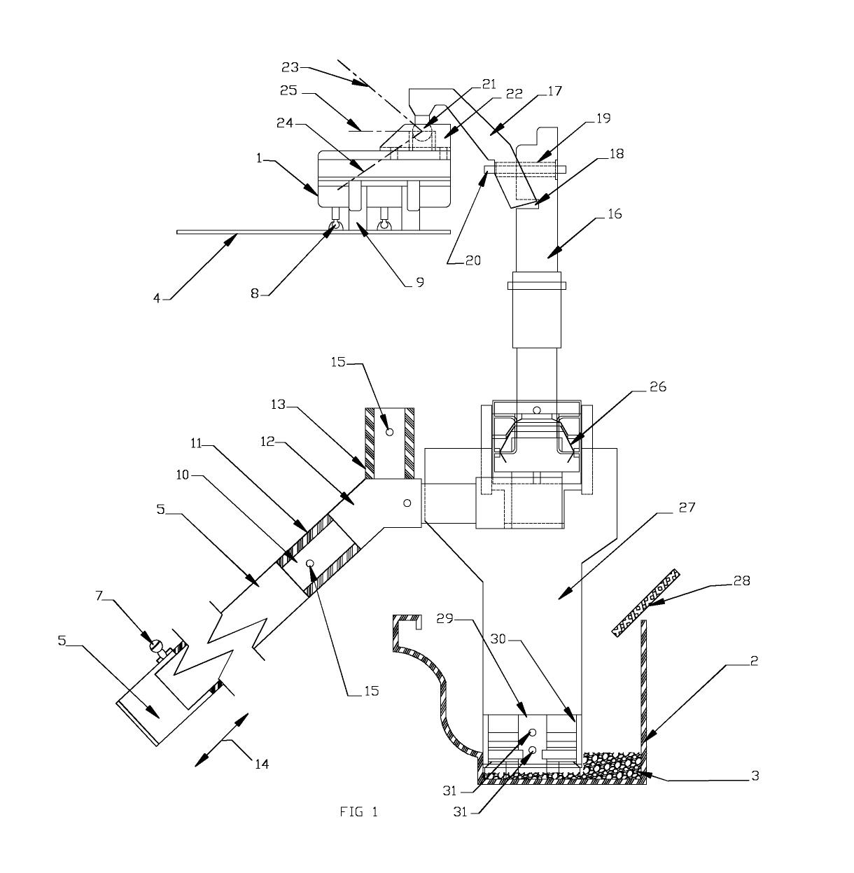

A power mirror attachment herein referred to as 1 is shown in FIG. 1, is used to inspect the gutter 2 for debris 3 by reflecting an image from the inside of the gutter 2 to the user on the ground. The image that is reflected by the reflective surface 4 of the power mirror attachment 1, will determine whether or not the gutter 2 needs to be cleaned.

First and foremost it must be said that before any inspection of the gutter 2 can be carried out, the telescoping electric pole 5 is used to place the scraper plate 6 into or above the gutter 2 to carry out the inspection process. If needed, the telescoping electric pole 5 can be extended to improve the reach of the power mirror attachment 1 and the scraper plate 6, by rotating and pulling on the extendible sections. To commence the inspection process, the user standing on the ground simply traverse the length of the gutter 2, while using the joystick 7, to select the optimum viewing angle of the reflective mirror surface 4, of the power mirror attachment 1. The joystick 7 is hard wired to the power mirror attachment 1. Once the joystick 7 is toggled in one of its many directions, it sends an electric signal via the telescoping electric pole 5 to the power mirror attachment 1. The signal received by the power mirror attachment 1, actuates the actuation leg 8 and the guide bar 9, which pushes down and pull up on the reflective surface 4 of the power mirror attachment 1. This allows the reflective surface 4 to tilt up or down, left to right, or front to back, until the optimum viewing angle is achieved. The said telescoping electric pole 5 is hollow with an externally threaded portion 10 that is shown screwed into an internally threaded head 1, herein referred to as 11, located on the neck 12. FIG. 1 also shows a second head 2 herein referred to as 13. Head 1 referred to as 11, is used for both inspection and cleaning. In FIG. 1, the telescoping direction 14 of the telescoping electric pole 7 is shown, and is parallel to the nominal position of the reflective surface 4 of the power mirror attachment 1. An anti-rotation pin 15 is shown passing through the neck 12, and the telescoping electric pole 5.

In FIG. 1, the hollow cylindrical vertical riser 16 is adjacent to the power mirror attachment 1. A cantilever arm 17 connects the hollow cylindrical vertical riser 16 and the power mirror attachment 1. There is a notch 18 in the hollow cylindrical vertical riser 16 that is used to locate the base of the cantilever arm 17. A compression spring 19 and cantilever adjustment screw 20 are located between the hollow cylindrical vertical riser 16 and the cantilever arm 17. When the cantilever adjustment screw 17 is torque to the right, the cantilever arm 17 tilts upwards by 5 degrees. Similarly, if the cantilever adjustment screw is torque to the left, the cantilever arm 17 will tilt downward by 5 degrees, from its nominal position. The notch 18 acts as a pivot point for the up and down movement of the cantilever arm 17. The ball joint 21 on the cantilever arm 17 is press fitted into a hole in the mirror retainer housing 22. The ball joint 21 provides manual adjustment of the entire power mirror attachment 1 in several directions with at least 3 degrees of freedom. FIG. 1 shows the power mirror attachment 1 upward tilt angle here in referred to as 23 and downward tilt angle referred to as 24. The maximum tilt angle from either side of the nominal position represented by 25 is 40 degrees.

In FIG. 1, there is a scraper return spring 26 that is used to return the scraper plate 27 to its original position after completing a cleaning cycle. The scraper plate 27 is used to remove debris 3, over the side of the gutter 2. The scraper plate 27 has a sleek slender design which provides adequate clearance to the roof shingle 28, in the cases where the roof shingle 28 protrude slightly over into the gutter 2, and into the path of the scraper plate 27.

In FIG. 1, there is a teeth assembly retainer plate 29, which is snapped fitted to the teeth assembly retainer 30 with the help of two (2) mechanical fasteners 31.

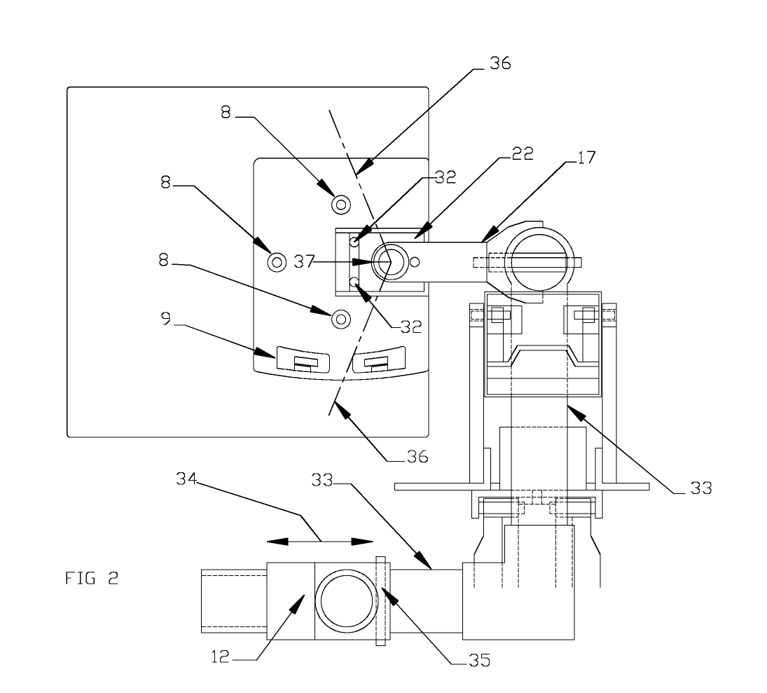

In FIG. 2, the mirror retainer housing 22 is attached to the top of the power mirror attachment 1 by two (2) mechanical fasteners 32. The neck 12 is connected to the hollow cylindrical horizontal base 33. The neck 12 as the ability to telescope horizontally as shown by the telescoping direction 34. The telescoping direction of the neck 12 is parallel to the cantilever arm. This additional degree of freedom enables the user to improve the leverage of the telescoping electric pole 5 during inspection and or cleaning. There is an anti-rotational pin 35 that passes through the neck 12 and the hollow cylindrical horizontal base 33 to prevent the neck 12 from rotating.

The ball joint 21 on the cantilever arm 17, allow the power mirror attachment 1 to rotate clockwise or counter clockwise, through an angular sweep 36, to either side of the central axis 37. In FIG. 2, there is a second internally threaded head 2 herein referred to as 13, on the neck 12 that is perpendicular to the hollow cylindrical horizontal base 33. Head 2 is also used with the telescoping electric pole 5 in the event that the apparatus is to be used for cleaning a gutter at the ground level.

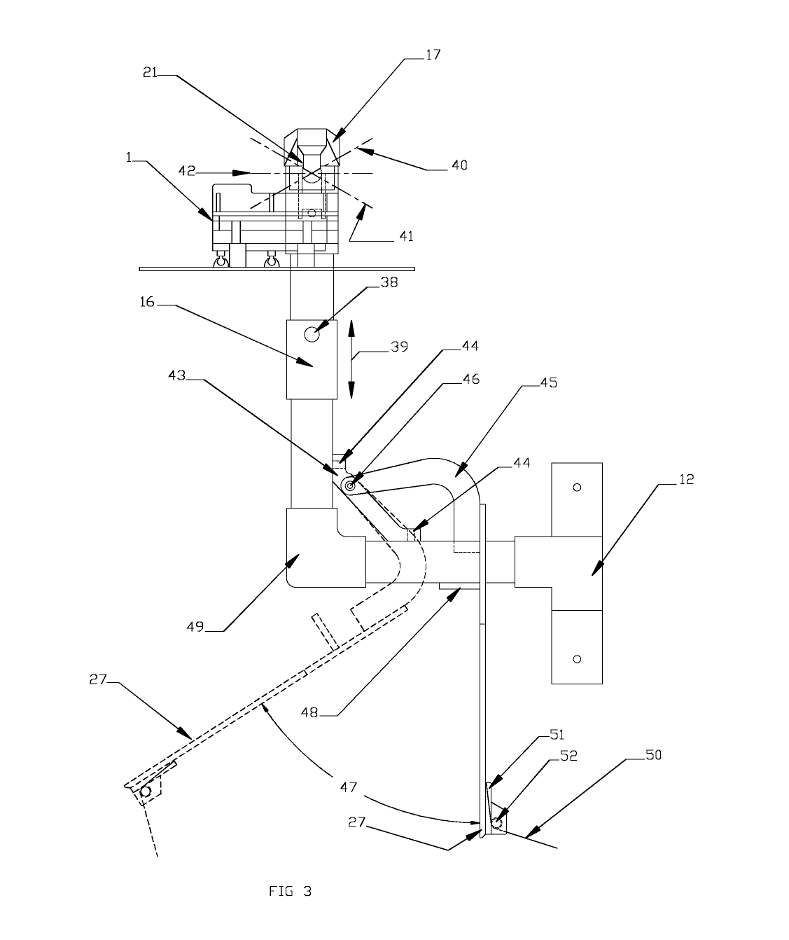

Moving onto FIG. 3, there is an adjustment locking pin 38 that allows the hollow cylindrical vertical riser 16 to telescope up and down, to increase the vertical height of the mirror. The telescoping direction is represented by 39. The adjustment locking pin 38 also serves as an anti-rotational pin. This telescoping ability of the hollow cylindrical vertical riser 16 provides the inspection and cleaning apparatus with 1 more degrees of freedom.

In FIG. 3, the power mirror attachment 1 can also tilt upwards represented by 40 or downwards represented by 41, at an angle of 30 degrees on either side of the center line 42. This capability allows the reflective mirror surface 4, to cover a wider area over which the debris 3 inside the gutter 2 can be viewed. Either one of the above mentioned position is achieved by simply holding the power mirror attachment 1 and articulating it about the ball joint 21 on the cantilever arm 17. The retainer housing bracket 43 is secured to the hollow cylindrical vertical riser 16 by two (2) mechanical fasteners 44.

In FIG. 3, the scraper arm 45 is pivoted about pins 46, which are located on the outside of the retainer housing bracket 43.

FIG. 3 shows the angular travel 47 of the scraper plate 27, as the user prepares to start a cleaning cycle. The scraper plate 27 travels through a maximum angle of 45 degrees during rotation.

There is a positive stop 48 which is used to prevent the scraper plate 27 from over rotating by making contact with the hollow cylindrical horizontal base 33. The hollow cylindrical horizontal base 33 is connected to the hollow cylindrical vertical riser 16 of the apparatus by means of a 90 degree elbow connector 49.

In FIG. 3, the scraper teeth 50 are shown assembled to the teeth assembly retainer 51. The scraper teeth 50 are pivoted about two (2) pins 52, located on the inside of the teeth assembly retainer 51.

In FIG. 3, the neck 12 is shown without the telescoping electric pole 5 attached.

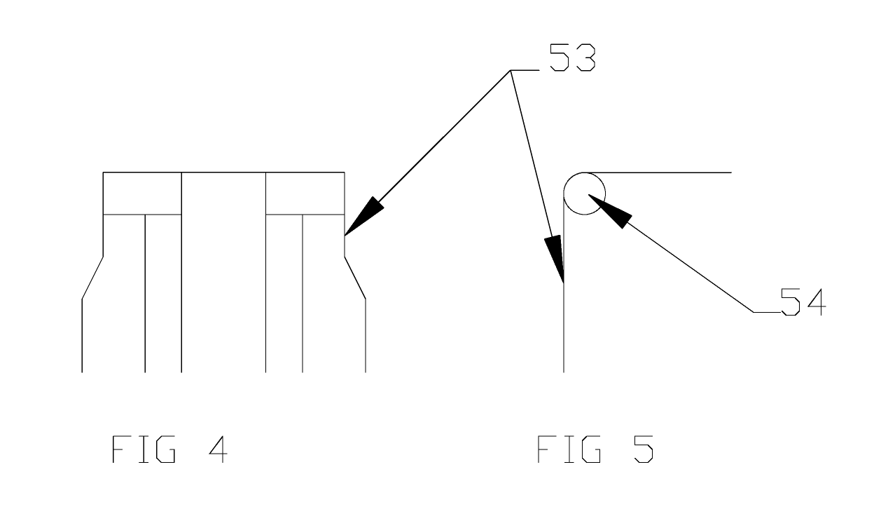

In FIG. 4, the scraper teeth assembly 53 is shown separately. The teeth assembly 53 is made from flexible steel wires coiled to form two (2) holes that provide the pivot points for rotation about the two (2) pins on the inside of the teeth assembly retainer 51.

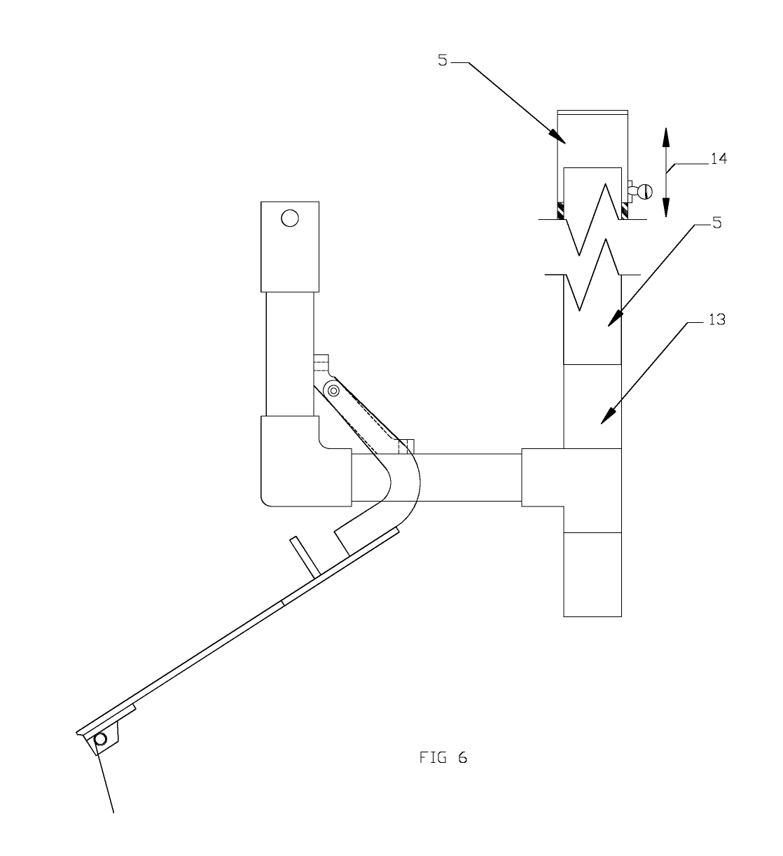

FIG. 5, shows the coil opening 54—details of the scraper teeth assembly 53.

FIG. 6, shows the apparatus in its cleaning position, with the telescoping electric pole 5 attached to head 2 herein referred to as 13. When using this set up, the user will simply telescope the pole by extending or shorten it, as represented by the telescoping direction 14. This position of the telescoping electric pole 5 is used when cleaning a gutter 2 at the ground level. Also, in FIG. 6 the power mirror attachment 1 as been disassembled front the apparatus, since it is not required during this cleaning mode.

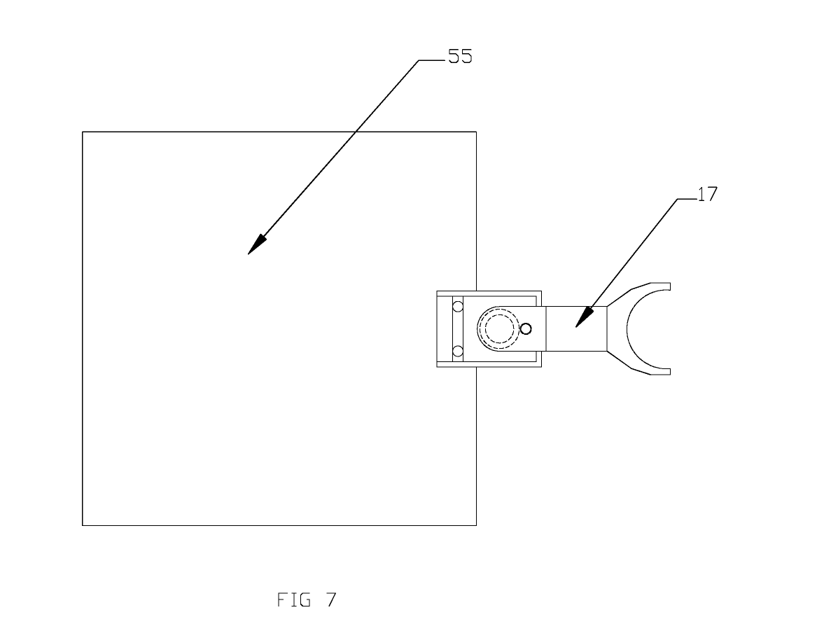

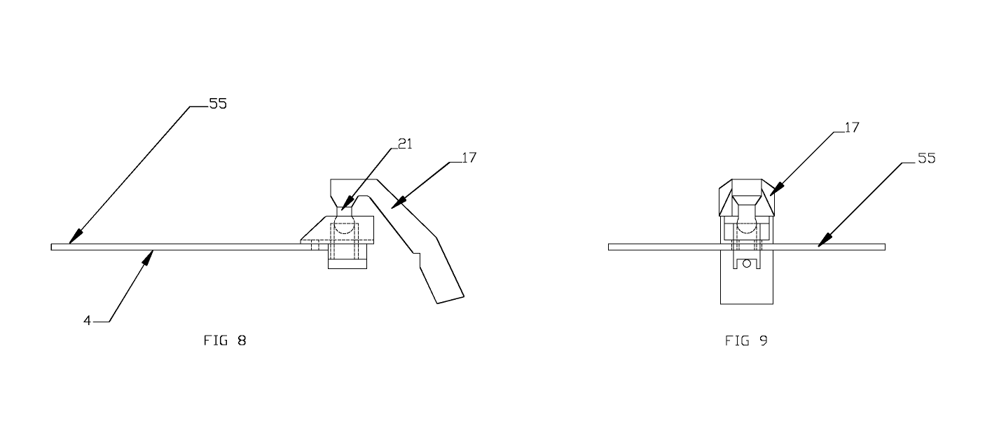

In FIGS. 7, 8 and 9 respectively, the non powered manual mirror attachment 55 is shown with the cantilever arm 17. To adjust the manual mirror attachment 55, the user simply holds the mirror reflective surface 4, and articulates it about the ball joint 21 on the cantilever arm 17.

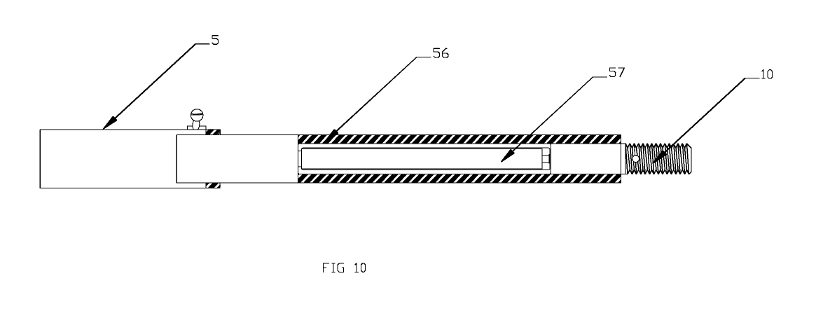

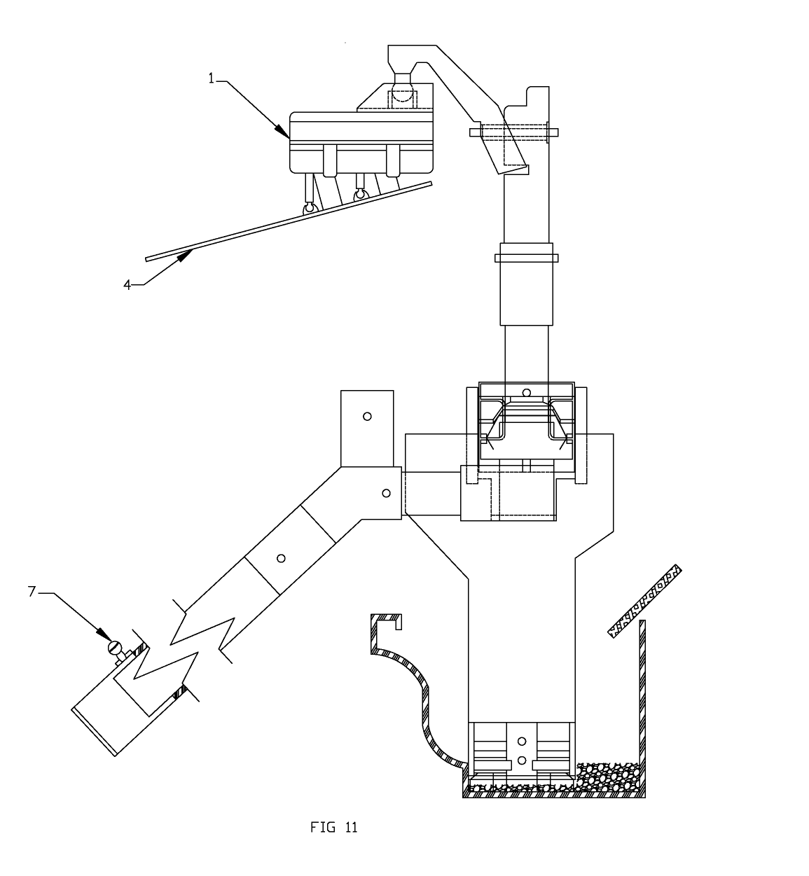

In FIG. 10, a section cut 56 is shown through the telescoping electric pole 5, making the 12V battery pack 57 visible. The 12V battery pack 57 is used to drive the power mirror attachment 1. The threaded end of the telescoping electric pole 5 is also shown.

In FIG. 11, the reflective surface of the power mirror attachment is tilted down to the left after the joystick 7 as been activated.

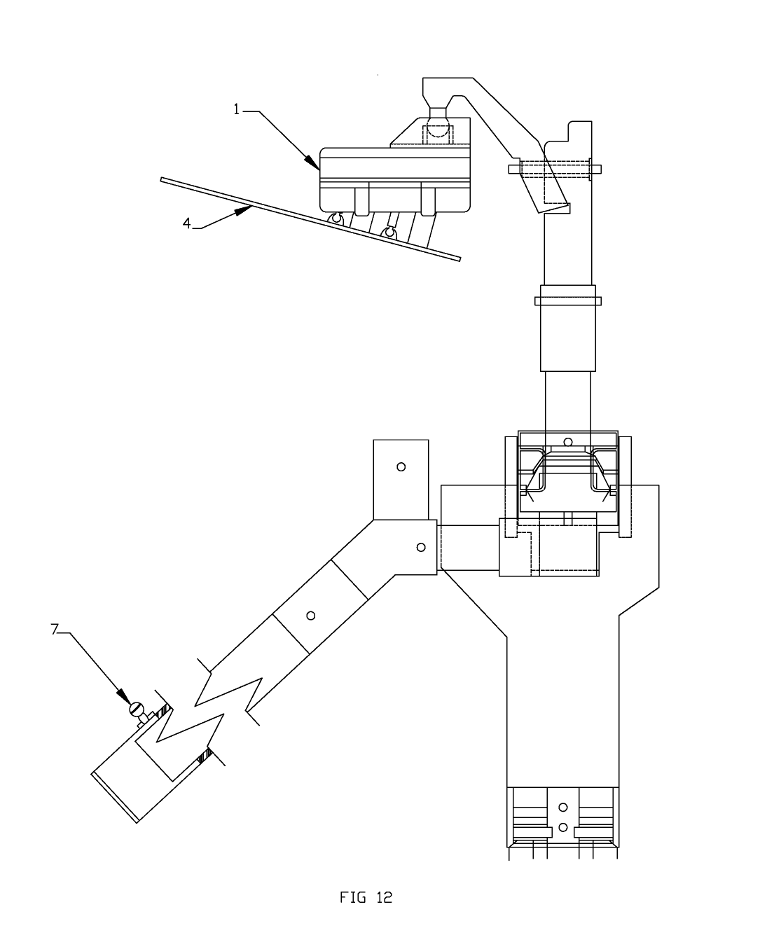

In FIG. 12, the reflective surface of the power mirror attachment is tilted up to the left after the joystick 7 as been activated.

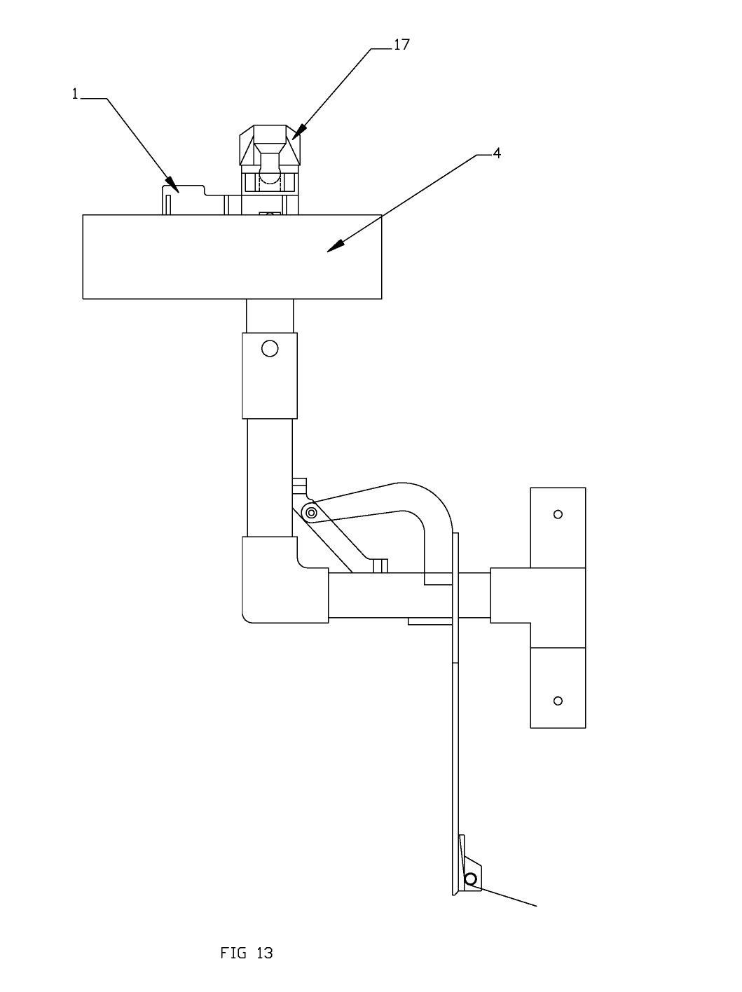

In FIG. 13, the reflective surface 4 of the power mirror attachment 1 is tilted up to the left putting the reflective surface 4 of the power mirror attachment 1 in clear view.

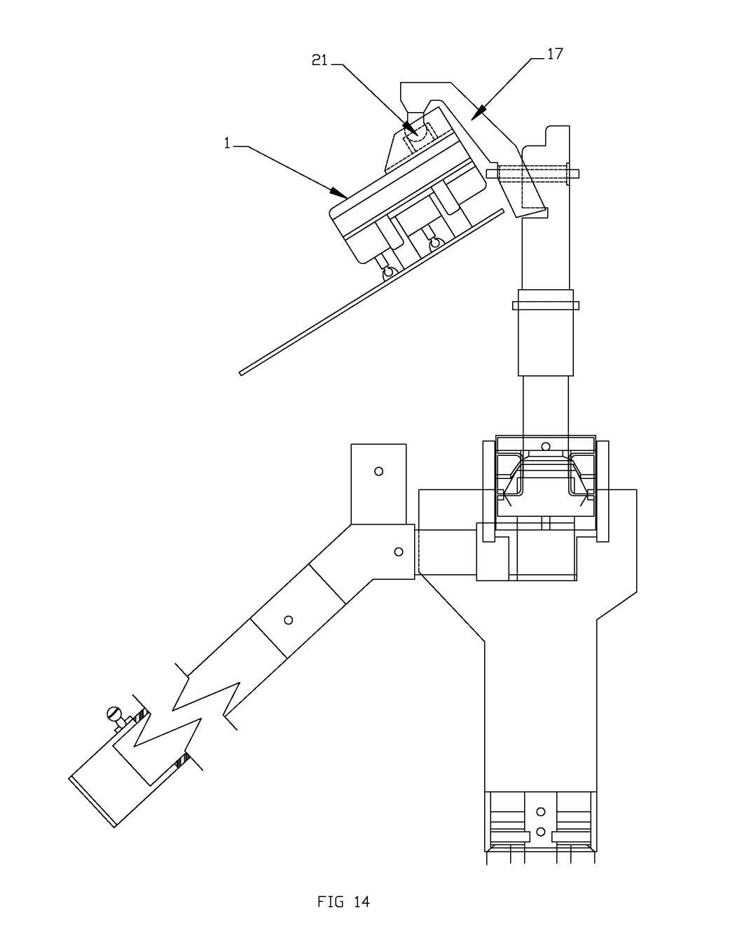

In FIG. 14, the power mirror attachment 1 as been manually tilted down to the left about the ball joint 21, located on the cantilever arm 17.

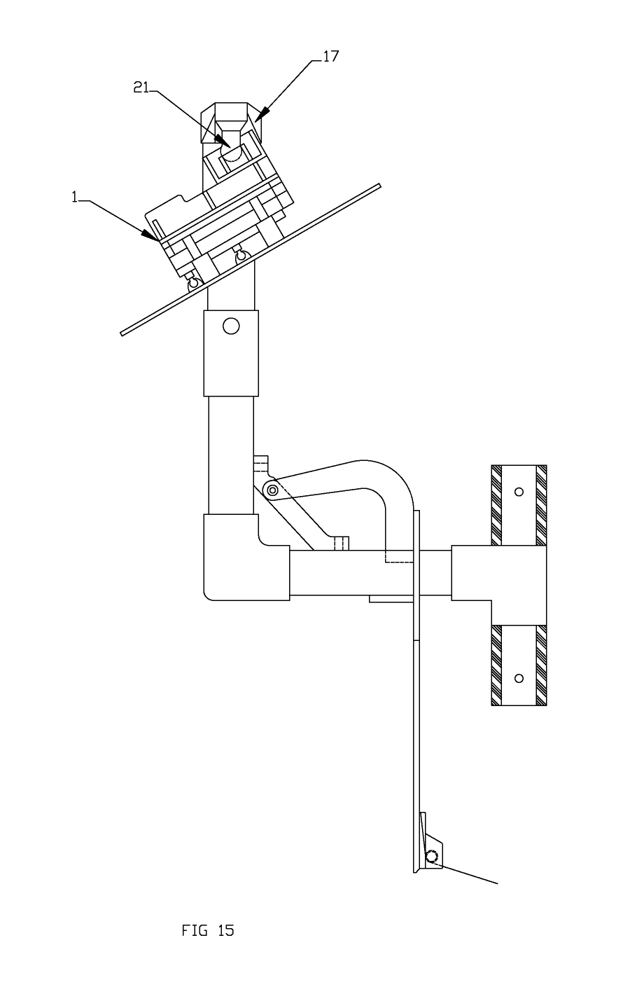

In FIG. 15, the power mirror attachment 1 as been manually tilted to the front about the ball joint 21, located on the cantilever arm 17.

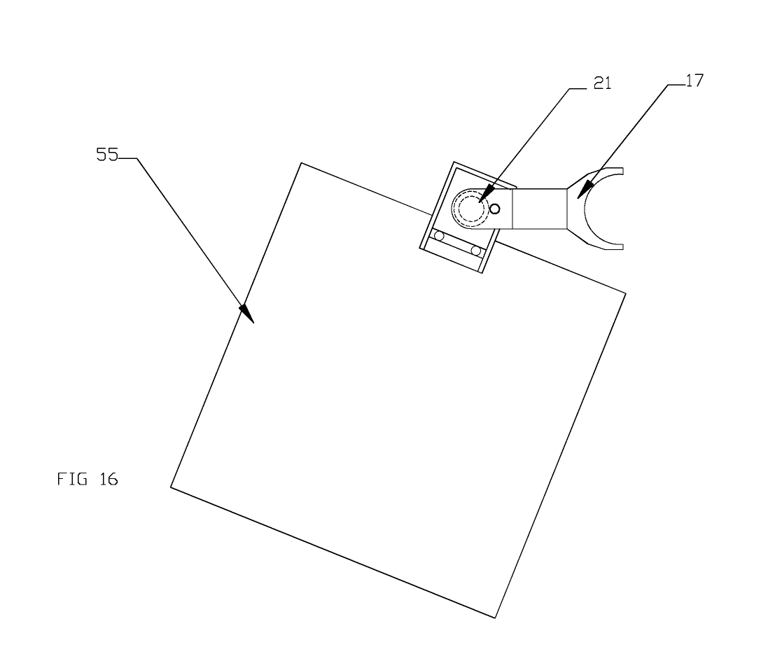

In FIG. 16, the manual mirror attachment 55 is shown rotated to the right. To rotate the manual mirror attachment 55, the user holds the mirror reflective surface 4, and rotates it about the ball joint 21 on the cantilever arm 17. The manual mirror attachment 55 can also be rotated to the right as required.

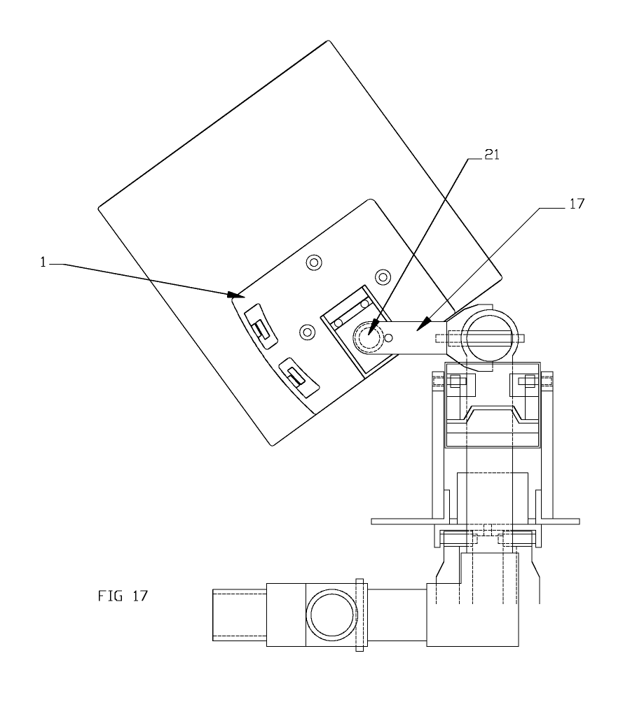

In FIG. 17, the power mirror attachment 1 is shown rotated to the left. The rotation of the power mirror attachment 1 is done in a similar manner to the manual mirror attachment 55. The user simply holds the power mirror reflective surface 4, and rotates it about the ball joint 21, on the cantilever arm 17. The power mirror attachment 1 can also be rotated to the right as required.

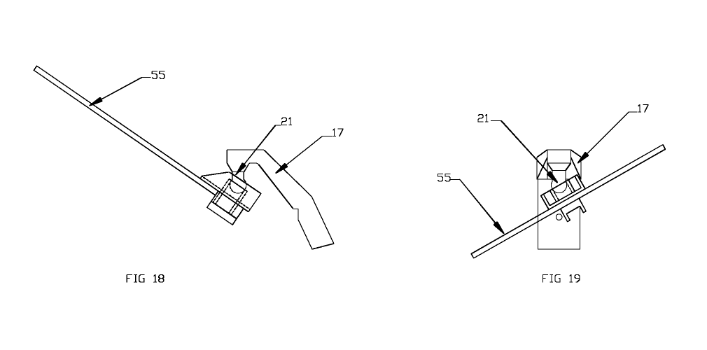

In FIG. 18, the manual mirror attachment 55 is shown tilted up to the left. To tilt the manual mirror attachment 55 upwards, the user holds the mirror reflective surface 4, and tilts it about the ball joint 21, on the cantilever arm 17. This adjustment is repeated for the downward position of the manual mirror attachment 55 as required.

In FIG. 19, the manual mirror attachment 55 is shown tilted up to the front. To tilt the manual mirror attachment 55, the user holds the mirror reflective surface 4, and tilts it about the ball joint 21, on the cantilever arm 17. If the manual mirror attachment 55 needs to be tilted to the right, the above procedure is repeated.

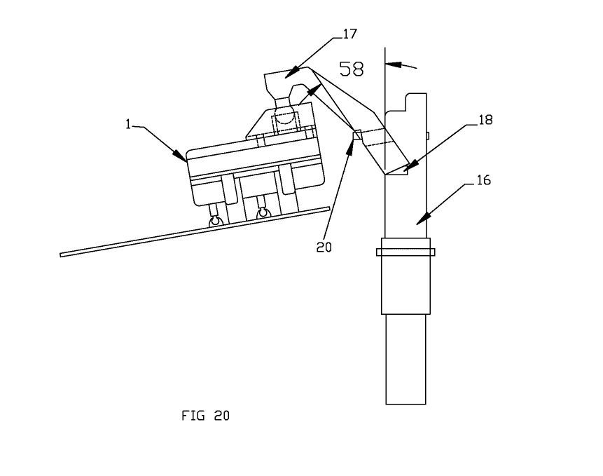

In FIG. 20, the cantilever arm 17 with the power mirror attachment is tilted downwards. The downward tilt angle is represented by the tilt angle 58. The cantilever arm 17 tilts about the notch 18 in the hollow cylindrical vertical riser 16, when the cantilever adjustment screw 19 is torque to the left. The cantilever arm 17 tilts upwards if the adjustment screw 19 is torque in the opposite direction.

All the rotational and tilt angles of either the power mirror attachment 1 or the manual mirror attachment 55, can be carried can be carried out in incremental amounts, about the ball joint 17.

1. A gutter inspection and cleaning apparatus, having the following components which are designed to inspect and clean a rain gutter.
An inspection component having;
a. Power mirror attachment mounted above the scraper mechanism to capture the entire travel of the scraper plate, as it travels from its rest position to its maximum working position.
b. Power mirror attachment having a flat reflective mirror surface which is used to reflect an image from inside the gutter back to the user, allowing the debris inside the gutter to be become visible.
c. Power mirror attachment which also as the ability to be adjusted manually in the event that there is a loss of power. The manual adjustment is carried out at the ball joint pivot point.
d. An optional non-power mirror attachment that as full manual adjustment.
e. Mirror retainer housing that is attached to the powered or non-powered mirror attachments by two (2) mechanical fasteners.
f. An articulating ball joint that is pivoted to the cantilever arm, which allows the said mirror retainer to be tilted up/down at a 45 degree angle, or at a 30 degree angle to the left and right about the central axis of the ball joint. The articulating ball joint also allows the mirror to be pivotally rotated about the central axis in the ball joint. The rotational angular travel of the mirror is up to 72 degrees, to either the left or right of the central axis, thus allowing the user to optimize the viewing angles for inspection and or cleaning. The total angular rotational travel of the mirror from left to right is 144+/−5 degrees.
g. A cantilever arm which connects the mirror attachment and the mirror retainer housing to the hollow cylindrical vertical riser.
h. A cantilever adjustment screw and compression spring located between the cantilever arm and the hollow cylindrical vertical riser. When the adjustment screw is torque to the left or right, it adjusts the tilt angle of the said cantilever arm and mirror up/down to optimize the viewing angles.
i. An adjustable hollow cylindrical vertical riser designed to adjust the height of the said mirror attachment up and down along a vertical axis with a locking pin.
j. The ability to inspect the gutter along its full length, which is achieved when the user moves the said apparatus longitudinally along a plane parallel to the length of the gutter. If the user determines that the gutter needs to be cleared, the said apparatus can now be used to clear the debris by means of the scraper mechanism.

2. A gutter inspection and cleaning apparatus, comprising of a scraper mechanism;
Scraper mechanism having;
a. A retainer support bracket mounted to a hollow cylindrical horizontal base that is connected to a hollow cylindrical vertical riser via a 90 degree elbow. The retainer support bracket is securely affixed to the hollow cylindrical base and hollow cylindrical vertical riser via two (2) mechanical fasteners, at an inclined angle.
b. A scraper plate return spring that is contained within the retainer support bracket. The spring is pivoted about two (2) pins located on the inside of the retainer bracket.
c. A scraper plate arm located below the power mirror attachment in claim 1, and is pivoted on two (2) pins located on the outside of the retainer support bracket. The scraper plate return spring is compressed between the retainer support bracket and the scraper plate arm.
d. A scraper plate is securely affixed to the scraper plate arm by a one (1) piece design molding process. The scraper plate rotates pivotally by means of the scraper plate arm through an acute angle of 37+/−2 degrees, when a force is applied perpendicular to the surface of the scraper plate. Once the scraper plate of the said apparatus is place inside the gutter normal to the cleaning surface, the user simply pull the inspection and cleaning apparatus forward, thus spring loading the scraper plate, at which point the trapped debris can be pulled out over the side of the gutter. Once the unbalanced force is removed from the scraper plate, is will return to its nominal position due the scraper plate return spring.
e. A retainer back plate that houses and secures the teeth assembly unto the scraper plate. The retainer back plate is affixed securely to the scraper plate by two (2) mechanical fasteners.
f. A teeth assembly located at end of the scraper plate for removing debris. The teeth assembly as six (6) total teeth made from flexible spring wire. The teeth assembly is pivotally located about two (2) pins on the inside of the retainer back plate.

3. A gutter inspection and cleaning apparatus, comprising of a carrier;
Carrier having;
a. A hollow cylindrical neck that is connected to the hollow cylindrical base by a 90 degree elbow connector. The neck as two (2) internally threaded receptors of standard thread, which allows it to accommodate either one of the two (2) poles mentioned below.
b. A hollow telescoping electric pole with a standard thread which is electrically wired for usage with the power mirror attachment.
c. An optional standard hollow telescoping pole for usage with the non-powered manual mirror attachment.
d. The said hollow cylindrical neck can be telescoped in and out to adjust the leverage of the inspection component as claimed in claim 1, and the scraper mechanism as claimed in claim 2. Thus improving the inspection and cleaning capability of the said inspection and cleaning apparatus.
e. An anti-rotational locking pin located between the hollow cylindrical neck and the hollow telescoping electric pole, connecting end.
f. A joystick is securely mounted to the said hollow telescoping electric pole for adjusting the viewing angles of the said power mirror remotely.
g. A 12V battery pack located inside of the hollow telescoping electric pole, which is used to drive the motor inside of the power mirror attachment.
h. The gutter inspection and cleaning apparatus as claimed in claim 1, is light weight and the components are made mostly of plastic materials.
